SEQUENCE LISTING (19 sequences)

SEQ ID NO: 1 - DNA, 1800 bp, Artificial Sequence
  GmSTK_IRAK
atgctgaattttcttaatttttctcttctattgacgctgttgtttcttctgtggaagcctgcgataagcaaaaacgggtgcacggataagtgtggacgcgttcgcattcaatttccattctacctcagaaataatagcaaattgaatcacaccaccaccgcctattcttctgggttcgatctgctatgtacggatgcagatgagactgtgctggagctgccttctgttccaataaaactgtttgtcaaaagcatagattacaagttgcagcaaatccaaatctacgaccctcaaaattgccttcccagggaactcataaaactcggcaattcctctgttgctccatttaaattccaatcatttggtgggaataatgtttccttcttccgctgcaactcaatgtcatcatcatgcccaattttgcaacttggatatgatgaaggtgactttattgatcctgaaataatatcttgcaccaaggtgagtgatgttttctccgtccagtggcaagtgaataattacttggggaacgcggtggttatggaatggtccaagcctgactgcagtacctgtgaagcacaagggcataaatgtaaatacaagaatggtactcaaggtgaaactgaatgtttcatttgcccaacaaacaggatttcaacatcaagtgttgttcttattgctgcagggggaatagttggaatgattcttttgctggtggtggtcaaggcgttgttgcatttgtatgaccattatatgacgaaaggagaagaccgggctcgaatggagaaattcttggaggattatagggcaatgaagcctactagattcacttatgctgatattaagagaatcacaaatgggtttagggaaagtttaggggaaggagctcatggagcagtcttcaaaggaatgctctcccgagaaattctcgttgccgtgaagatactcaatgacacagtgggagatggaaaggatttcataaacgaagtgggaaccatgggcaaaattcatcatgttaacgttgttcgcttgcttggattctgtgcagatggattccaccgcgctctcgtctatgatttcttccctaacggatcactgcagagattcttggctccaccggacaacaaggatgttttccttggttgggagaagttgcaacaaattgctcttggtgttgccaaaggggttgagtatctccaccttggctgtgatcaaagaataattcactttgacatcaatcctcacaatattttaatagatgaccattttgttccaaaaatcactgattttggacttgccaagttgtgtcccaaaaatcaaagtacagtttctataactgctgctaggggaaccttaggctacattgctcctgaagttttctcaagaaactttggtaatgtttcttataagtctgacatttatagttatggaatgttgctcttagagatggtgggaggaagaaagaatacaaatatgtcagcagaggaaagtttccaagttttgtaccctgaatggatccataatttgctcaaaagcagagacgtgcaagttactattgaggatgagggagatgttagaattgccaagaaacttgccattgtaggactttggtgcattgagtggaacccaatagaccgtccatccatgaaaactgtgatacaaatgcttgaaggagatggagacaagttaattgcaccacctactccttttgacaagaccagctcttctagaacaagtgtagttgctccaacaagacgccagaattttgagttggaaattattaatgaaatagaagaataa

SEQ ID NO: 2 - protein, 599 aa, Artificial Sequence
  GmSTK_IRAK
MLNFLNFSLLLTLLFLLWKPAISKNGCTDKCGRVRIQFPFYLRNNSKLNHTTTAYSSGFDLLCTDADETVLELPSVPIKLFVKSIDYKLQQIQIYDPQNCLPRELIKLGNSSVAPFKFQSFGGNNVSFFRCNSMSSSCPILQLGYDEGDFIDPEIISCTKVSDVFSVQWQVNNYLGNAVVMEWSKPDCSTCEAQGHKCKYKNGTQGETECFICPTNRISTSSVVLIAAGGIVGMILLLVVVKALLHLYDHYMTKGEDRARMEKFLEDYRAMKPTRFTYADIKRITNGFRESLGEGAHGAVFKGMLSREILVAVKILNDTVGDGKDFINEVGTMGKIHHVNVVRLLGFCADGFHRALVYDFFPNGSLQRFLAPPDNKDVFLGWEKLQQIALGVAKGVEYLHLGCDQRIIHFDINPHNILIDDHFVPKITDFGLAKLCPKNQSTVSITAARGTLGYIAPEVFSRNFGNVSYKSDIYSYGMLLLEMVGGRKNTNMSAEESFQVLYPEWIHNLLKSRDVQVTIEDEGDVRIAKKLAIVGLWCIEWNPIDRPSMKTVIQMLEGDGDKLIAPPTPFDKTSSSRTSVVAPTRRQNFELEIINEIEE

SEQ ID NO: 3 - DNA, 1801 bp, Artificial Sequence
  GmSTK_IRAK gene-silenced transgenic offsprings
atgctgaattttcttaatttttctcttctattgacgctgttgtttcttctgtggaagcctgcgataagcaaaaacgggtgcacggataagtgtggacgcgttcgcattcaatttccattctacctcagaaataatagcaaattgaatcacaccaccaccgcctattcttctgggttcgatctgctatgtacggatgcagatgagactgtgctggagctgccttctgttccaataaaactgtttgtcaaaagcatagattacaagttgcagcaaatccaaatctacgaccctcaaaattgccttcccagggaactcataaaactcggcaattcctctgttgctccatttaaattccaatcatttggtgggaataatgtttccttcttccgctgcaactcaatgtcatcatcatgcccaattttgcaacttggatatgatgaaggtgactttattgatcctgaaataatatcttgcaccaaggtgagtgatgttttctccgtccagtggcaagtgaataattacttggggaacgcggtggttatggaatggtccaagcctgactgcagtacctgtgaagcaacaagggcataaatgtaaatacaagaatggtactcaaggtgaaactgaatgtttcatttgcccaacaaacaggatttcaacatcaagtgttgttcttattgctgcagggggaatagttggaatgattcttttgctggtggtggtcaaggcgttgttgcatttgtatgaccattatatgacgaaaggagaagaccgggctcgaatggagaaattcttggaggattatagggcaatgaagcctactagattcacttatgctgatattaagagaatcacaaatgggtttagggaaagtttaggggaaggagctcatggagcagtcttcaaaggaatgctctcccgagaaattctcgttgccgtgaagatactcaatgacacagtgggagatggaaaggatttcataaacgaagtgggaaccatgggcaaaattcatcatgttaacgttgttcgcttgcttggattctgtgcagatggattccaccgcgctctcgtctatgatttcttccctaacggatcactgcagagattcttggctccaccggacaacaaggatgttttccttggttgggagaagttgcaacaaattgctcttggtgttgccaaaggggttgagtatctccaccttggctgtgatcaaagaataattcactttgacatcaatcctcacaatattttaatagatgaccattttgttccaaaaatcactgattttggacttgccaagttgtgtcccaaaaatcaaagtacagtttctataactgctgctaggggaaccttaggctacattgctcctgaagttttctcaagaaactttggtaatgtttcttataagtctgacatttatagttatggaatgttgctcttagagatggtgggaggaagaaagaatacaaatatgtcagcagaggaaagtttccaagttttgtaccctgaatggatccataatttgctcaaaagcagagacgtgcaagttactattgaggatgagggagatgttagaattgccaagaaacttgccattgtaggactttggtgcattgagtggaacccaatagaccgtccatccatgaaaactgtgatacaaatgcttgaaggagatggagacaagttaattgcaccacctactccttttgacaagaccagctcttctagaacaagtgtagttgctccaacaagacgccagaattttgagttggaaattattaatgaaatagaagaataa

SEQ ID NO: 4 - DNA, 1798 bp, Artificial Sequence
  GmSTK_IRAK gene-silenced transgenic offsprings
atgctgaattttcttaatttttctcttctattgacgctgttgtttcttctgtggaagcctgcgataagcaaaaacgggtgcacggataagtgtggacgcgttcgcattcaatttccattctacctcagaaataatagcaaattgaatcacaccaccaccgcctattcttctgggttcgatctgctatgtacggatgcagatgagactgtgctggagctgccttctgttccaataaaactgtttgtcaaaagcatagattacaagttgcagcaaatccaaatctacgaccctcaaaattgccttcccagggaactcataaaactcggcaattcctctgttgctccatttaaattccaatcatttggtgggaataatgtttccttcttccgctgcaactcaatgtcatcatcatgcccaattttgcaacttggatatgatgaaggtgactttattgatcctgaaataatatcttgcaccaaggtgagtgatgttttctccgtccagtggcaagtgaataattacttggggaacgcggtggttatggaatggtccaagcctgactgcagtacctgtgaagcaagggcataaatgtaaatacaagaatggtactcaaggtgaaactgaatgtttcatttgcccaacaaacaggatttcaacatcaagtgttgttcttattgctgcagggggaatagttggaatgattcttttgctggtggtggtcaaggcgttgttgcatttgtatgaccattatatgacgaaaggagaagaccgggctcgaatggagaaattcttggaggattatagggcaatgaagcctactagattcacttatgctgatattaagagaatcacaaatgggtttagggaaagtttaggggaaggagctcatggagcagtcttcaaaggaatgctctcccgagaaattctcgttgccgtgaagatactcaatgacacagtgggagatggaaaggatttcataaacgaagtgggaaccatgggcaaaattcatcatgttaacgttgttcgcttgcttggattctgtgcagatggattccaccgcgctctcgtctatgatttcttccctaacggatcactgcagagattcttggctccaccggacaacaaggatgttttccttggttgggagaagttgcaacaaattgctcttggtgttgccaaaggggttgagtatctccaccttggctgtgatcaaagaataattcactttgacatcaatcctcacaatattttaatagatgaccattttgttccaaaaatcactgattttggacttgccaagttgtgtcccaaaaatcaaagtacagtttctataactgctgctaggggaaccttaggctacattgctcctgaagttttctcaagaaactttggtaatgtttcttataagtctgacatttatagttatggaatgttgctcttagagatggtgggaggaagaaagaatacaaatatgtcagcagaggaaagtttccaagttttgtaccctgaatggatccataatttgctcaaaagcagagacgtgcaagttactattgaggatgagggagatgttagaattgccaagaaacttgccattgtaggactttggtgcattgagtggaacccaatagaccgtccatccatgaaaactgtgatacaaatgcttgaaggagatggagacaagttaattgcaccacctactccttttgacaagaccagctcttctagaacaagtgtagttgctccaacaagacgccagaattttgagttggaaattattaatgaaatagaagaataa

SEQ ID NO: 5 - DNA, 31 bp, Artificial Sequence
  GmSTK_IRAK-F
cctaccacattaattactcactcttcactca

SEQ ID NO: 6 - DNA, 29 bp, Artificial Sequence
  GmSTK_IRAK-R
tcaactttaacgctcattcctgcattcat

SEQ ID NO: 7 - DNA, 38 bp, Artificial Sequence
  upstream primer used for the seamless cloning
tttggagagaacacgtatggctgagcttcactaccaac

SEQ ID NO: 8 - DNA, 38 bp, Artificial Sequence
  downstream primer used for the seamless cloning
tcggggaaattcggggttaagaagcctgcaccccactg

SEQ ID NO: 9 - DNA, 20 bp, Artificial Sequence
  gRNA
gcagtacctgtgaagcacaa

SEQ ID NO: 10 - DNA, 20 bp, Artificial Sequence
  upstream primer of the EPSPS gene
aggacgtcatcaatacgggc

SEQ ID NO: 11 - DNA, 20 bp, Artificial Sequence
  downstream primer of the EPSPS gene
atccacgccattgagcttga

SEQ ID NO: 12 - DNA, 18 bp, Artificial Sequence
  upstream primer of the bar gene
atgagcccagaacgacgc

SEQ ID NO: 13 - DNA, 19 bp, Artificial Sequence
  downstream primer of the bar gene
acgtcatgccagttcccgt

SEQ ID NO: 14 - DNA, 22 bp, Artificial Sequence
  upstream primer
gcagcaaatccaaatctacgac

SEQ ID NO: 15 - DNA, 23 bp, Artificial Sequence
  downstream primer
cggtcttctcctttcgtcatata

SEQ ID NO: 16 - DNA, 28 bp, Artificial Sequence
  WT - wild-type target sequence
gcagtacctgtgaagcacaagggcataa

SEQ ID NO: 17 - DNA, 29 bp, Artificial Sequence
  KO-1 - a nucleotide sequence of GmSTK_IRAK
gcagtacctgtgaagcaacaagggcataa

SEQ ID NO: 18 - DNA, 26 bp, Artificial Sequence
  KO-2 - a nucleotide sequence of GmSTK_IRAK
gcagtacctgtgaagcaagggcataa

SEQ ID NO: 19 - DNA, 26 bp, Artificial Sequence
  KO-3 - a nucleotide sequence of GmSTK_IRAK
gcagtacctgtgaagcaagggcataa

(19) United States
(12) Patent Application Publication
(10) Pub. No.: US 2022/0162633 A1
(43) Pub. Date: May 26, 2022

(54) USE OF SOYBEAN PROTEIN KINASE GENE GMSTK_IRAK

(71) Applicant: Henan Agricultural University, Zhengzhou City (CN)

(72) Inventors: Dan ZHANG, Zhengzhou City (CN); Xingguo ZHANG, Zhengzhou City (CN); Yuming YANG, Zhengzhou City (CN); Haiyan LV, Zhengzhou City (CN); Shanshan CHU, Zhengzhou City (CN)

(21) Appl. No.: 17/223,782

(22) Filed: Apr. 6, 2021

(30) Foreign Application Priority Data
Nov. 24, 2020 (CN) 202011330937.7

(51) Int. Cl. C12N 15/82; C12N 9/22

(52) U.S. Cl. CPC C12N 15/8265; C12N 9/22

(57) ABSTRACT

The present disclosure provides use of a soybean protein kinase gene GmSTK_IRAK and belongs to the technical field of plant genetic engineering. PCR is used to clone the soybean GmSTK_IRAK gene, a transgene and gene editing technology is used to obtain GmSTK_IRAK-over-expressing transgenic plants and GmSTK_IRAK gene-silenced transgenic plants. GmSTK_IRAK-over-expressing soybeans can greatly improve phosphorus absorption and utilization efficiency, biomass and yield, while GmSTK_IRAK gene-silenced soybeans can reduce phosphorus absorption and utilization efficiency, biomass and yield. The soybean protein kinase gene GmSTK_IRAK can be used as a target gene to be introduced into plants to regulate the balance of phosphorus metabolism in transgenic plants, and is of great significance for cultivating new soybean varieties with high phosphorus efficiency.

### TECHNICAL FIELD

The present disclosure belongs to the technical field of plant genetic engineering, and particularly relates to use of a soybean protein kinase gene GmSTK_IRAK.

BACKGROUND

Soybeans are a crop that requires a large amount of phosphorus, and its grain phosphorus content is much higher than that of rice, wheat, corn and other crops (Li Xihuan et al., 2011). Phosphorus deficiency not only affects the growth and development of soybeans, but also significantly affects the formation of root nodules, and finally badly affects the yield (Wang Shuqi et al., 2010). Phosphorus deficiency in the soil has become an important factor limiting soybean yield in our country (Liu Haixu et al., 2017). In order to solve the contradiction between high phosphorus requirement of soybeans and low available phosphorus in soil, isolating and identifying new key genes of low phosphorus tolerance, introducing low phosphorus sensitive varieties by means of genetic engineering, and carrying out high efficiency molecular breeding of phosphorus are effective methods to improve low phosphorus tolerance ability and international competitiveness of soybean.

Interleukin-1 receptor associated kinases (IRAK) are a type of serine/threonine kinases (STK) related to signal transduction and play an important role in regulation of inflammatory responses of humans and mammals and protective reactions to pathogens (Oliveira et al., 2011; Singer et al., 2018). In rice, a homologous gene PSTOL1 (58%) of GmSTK_IRAK can be used as an early root growth promoter and enable plants to obtain more phosphorus and other nutrients (Gamuyao et al., 2012). In Arabidopsis thaliana, GmSTK_IRAK homologous gene encoded PR5K 22 (58%) and SNC4 have been reported to be involved in plant resistance to adversity stress (Cheng et al., 2011; Zhang et al., 2014; Baek et al. , 2019). However, researches of GmSTK_IRAK gene in soybeans have not been reported so far.

### SUMMARY

In view of this, the present disclosure is intended to solve the contradiction between high phosphorus requirement of soybeans, and global phosphorus resource crisis and low available phosphorus in soil, and provide use of gene GmSTK_IRAK in improving phosphorus absorption and utilization efficiency of soybeans, improving absorption and utilization of phosphorus, thus achieving phosphorus-efficient breeding and improving soybean yield and competitiveness.

To achieve the foregoing purpose, the present disclosure provides the following technical solution:

The present disclosure provides use of a soybean protein kinase gene GmSTK_IRAK in improving phosphorus absorption and utilization efficiency of soybeans.

Preferably, the soybean protein kinase gene GmSTK_IRAK may have a nucleotide sequence shown in SEQ ID NO. 1 and a protein encoded by the soybean protein kinase gene GmSTK_IRAK may have an amino acid sequence shown in SEQ ID NO. 2.

Preferably, the GmSTK_IRAK gene may be over-expressed or silenced in soybean plants.

Preferably, the over-expression may include introducing the soybean protein kinase gene GmSTK_IRAK into a target plant through a recombinant plasmid.

Preferably, the recombinant plasmid may be a plant over-expressing vector containing the soybean protein kinase gene GmSTK_IRAK.

Preferably, the plant over-expressing vector may be DTS6004.

Preferably, the GmSTK_IRAK gene in soybeans may be silenced through a gene editing technology, and specifically, a Cas9 gene editing technology may be used to edit the GmSTK_IRAK gene to obtain GmSTK_IRAK gene-silenced transgenic offsprings.

Preferably, the GmSTK_IRAK gene-silenced transgenic offsprings may have a nucleotide sequence shown in SEQ ID NO. 3 and/or SEQ ID NO. 4.

The present disclosure also provides use of a recombinant vector and cell containing a soybean protein kinase gene GmSTK_IRAK in improving phosphorus absorption and utilization efficiency of soybeans.

The present disclosure also provides use of a soybean protein kinase gene GmSTK_IRAK and a recombinant vector and cell thereof in breeding and cultivating soybeans.

Beneficial effects of the present disclosure are as follows:

The present disclosure finds that an expression amount of GmSTK_IRAK in soybeans is significantly positively correlated with phosphorus absorption and utilization efficiency of soybeans. The GmSTK_IRAK gene is over-expressed or silenced in soybeans, and can change root configurations of transgenic soybeans and regulate phosphorus absorption and utilization efficiency, biomass and yield. The expression amount of the soybean protein kinase gene GmSTK_IRAK can be regulated by a biotechnological means, thus new soybean varieties with high phosphorus absorption and utilization are cultivated, and the soybean protein kinase gene GmSTK_IRAK has good application prospects.

This application references material in the ASCII text file, titled SEQUENCE_LISTING_JSD_20210506, created May 6, 2021,size 15,914 bytes, the disclosure of which is incorporated by reference herein for all purposes.

### BRIEF DESCRIPTION OF THE DRAWINGS

FIG. 1 is a diagram of a GmSTK_IRAK gene-over-expressing vector, where, the vector contains an EPSPS gene as a transgenic selection marker and the DTS6004-GmSTK_IRAK vector uses a 35S promoter followed by a full-length GmSTK_IRAK cDNA and can be used for transgene to obtain GmSTK_IRAK-over-expressing transgenic plants;

FIG. 2 is a diagram of a GmSTK_IRAK gene-silenced vector, where, the vector contains a Bar gene as a transgenic selection marker, uses an AtU6 promoter followed by a Cas9 target sequence and a Cas9 gene and can be used for transgene to obtain GmSTK_IRAK-silenced transgenic plants;

FIG. 3 shows verification results of positive transgenic soybean plants, where A is a PCR verification of a GmSTK_IRAK gene in soybeans, B is a PCR verification of the EPSPS gene in soybeans, M is a 2,000 bp marker, C is a blank control, + is a plasmid positive control, and 1-10 represent samples of independent transgenic lines 1-10, respectively;

FIG. 4 shows types of GmSTK_IRAK gene editing, where WT is a wild-type target sequence; and KO-1, KO-2, and KO-3 represent sequences of three different edited transgenic lines;

FIG. 5 is a phenotype identification of GmSTK_IRAK transgenic offsprings (T4), where NPCK1-1, NPCK1-2 and NPCK1-3 represent wild-type controls at normal phosphorus levels; NPOE-1, NPOE-2 and NPOE-3 represent different over-expressing transgenic lines at normal phosphorus levels; LPCK1-1, LPCK1 -2 and LPCK1-3 represent wild-type controls at low phosphorus levels; LPOE-1, LPOE-2 and LPOE-3 represent different over-expressing transgenic lines at low phosphorus levels; NPCK2-1, NPCK2-2 and NPCK2-3 represent wild-type controls at normal phosphorus levels; NPKO-1, NPKO-2 and NPKO-3 represent different silenced transgenic lines at normal phosphorus levels; LPCK2-1, LPCK2-2 and LPCK2-3 represent wild-type controls at low phosphorus levels; and LPKO-1, LPKO-2 and LPKO-3 represent different silenced transgenic lines at low phosphorus levels;

FIG. 6 shows root system architectures of GmSTK_IRAK transgenic offsprings (T4), where NP represents a normal phosphorus level and LP represents a low phosphorus level; OE-CK and KO-CK represent different wild types; OE-1, OE-2 and OE-3 represent different GmSTK_IRAK-over-expressing transgenic lines; and KO-1, KO-2 and KO-3 represent different GmSTK_IRAK-silenced transgenic lines;

FIG. 7 is phosphorus absorption efficiency (PAE) of GmSTK_IRAK transgenic offsprings (T4), where NP represents a normal phosphorus level and LP represents a low phosphorus level; OE-CK and KO-CK represent wild types separately; OE-1, OE-2 and OE-3 represent different GmSTK_IRAK-over-expressing transgenic lines; KO-1, KO-2 and KO-3 represent different GmSTK_IRAK-silenced transgenic lines; and “root” represents roots and “shoot” represents an overground part;

FIG. 8 shows yield traits of GmSTK_IRAK transgenic offsprings (T4), where NP represents a normal phosphorus level and LP represents a low phosphorus level; OE-CK and KO-CK represent different wild types; OE-1, OE-2 and OE-3 represent different GmSTK_IRAK-over-expressing transgenic lines; and KO-1, KO-2 and KO-3 represent different GmSTK_IRAK-silenced transgenic lines.

### DETAILED DESCRIPTION

The present disclosure provides use of a soybean protein kinase gene GmSTK_IRAK in improving phosphorus absorption and utilization efficiency of soybeans. Preferably, the soybean protein kinase gene GmSTK_IRAK may have a nucleotide sequence shown in SEQ ID NO. 1 and a protein encoded by the soybean protein kinase gene GmSTK_IRAK may have an amino acid sequence shown in SEQ ID NO. 2.

When a soybean protein kinase gene GmSTK_IRAK is applied to improve soybean phosphorus absorption and utilization efficiency, the present disclosure includes a step of transforming a plant expression vector carrying the GmSTK_IRAK into a plant cell or tissue. The present disclosure has no special limitations on the transformation method, where, preferably, a biotransformation method may be used to transform the plant cell or tissue. The present disclosure has no special limitations on the biotransformation method. For example, conventional biological methods such as direct DNA transformation, microinjection, electrical conduction and agrobacterium-mediating may be used to transform the plant cell or tissue. The present disclosure has no special limitations on types of the plant expression vectors, and any vector that can guide an expression of foreign genes in plants can be used, such as a Ti plasmid, a Ri plasmid, a plant virus vector and the like. In specific examples of the present disclosure, when the GmSTK_IRAK gene is over-expressed in soybean plants, the protein kinase gene GmSTK_IRAK may be preferably introduced into a target plant through a recombinant plasmid, the recombinant plasmid may preferably be a plant over-expressing vector containing the soybean protein kinase gene GmSTK_IRAK and the plant over-expressing vector may preferably be DTS6004.

When a GmSTK_IRAK gene of the present disclosure is used to construct a plant expression vector, any enhanced promoter or inducible promoter can be added before a transcription initiation nucleotide of the GmSTK_IRAK gene. In order to facilitate identification and screening of a transgenic plant cell or plant, the used plant expression vector can be processed, for example, selectable marker genes (a GUS gene, a luciferase gene and the like) expressed in plants or antibiotic markers with resistance (a gentamicin marker, a kanamycin marker and the like) can be added. Considering safety of transgenic plants, it is also possible to directly screen transformed plants by phenotypes without adding any selectable marker genes.

In specific examples of the present disclosure, when the GmSTK_IRAK gene is silenced in soybean plants, there is no special limitations on a specific method of silencing, preferably, the GmSTK_IRAK gene may be silenced in soybeans by a gene editing technology, more preferably, a Cas9 gene editing technology may edit the GmSTK_IRAK to obtain GmSTK_IRAK gene-silenced transgenic offsprings, and preferably, the GmSTK_IRAK gene-silenced transgenic offsprings may have nucleotide sequences shown in SEQ ID NO. 3 and/or SEQ ID NO. 4.

The present disclosure also provides use of a recombinant vector and cell containing a soybean protein kinase gene GmSTK_IRAK in improving plant phosphorus absorption and utilization efficiency of soybeans. The present disclosure has no special limitations on types of recombinant vectors and cells. Conventional recombinant vectors and cells in the field may be used. The recombinant vector containing the soybean protein kinase gene GmSTK_IRAK can transform a host, and the transformed host may be a monocot plant and also a dicotyledonous plant.

The present disclosure also provides use of a soybean protein kinase gene GmSTK_IRAK and a recombinant vector and cell thereof in soybean breeding and cultivation.

Unless defined otherwise, all technical and scientific terms used herein have the same meanings as commonly understood by one of ordinary skill in the art. The technical solutions provided by the present disclosure will be described in detail below with reference to examples, but the examples should not be construed as limiting the claimed scope of the present disclosure. In the following examples, various processes and methods that are not described in detail are conventional methods known in the art. The primers used are all marked when they appear for the first time, and the same primers used thereafter are all the same as those marked for the first time. Methods used in the following examples are conventional methods unless otherwise specified.

### EXAMPLE 1

### Cloning of Soybean GmSTK_IRAK Gene and Construction of Plant Expression Vector

### (1) Primer Design, RNA Extraction and cDNA Reverse Transcription

A plant total RNA extraction kit (DP432, Tiangen) was used to extract total RNA of soybean Williams 82 leaves, and integrity of the RNA was detected by 1% agarose gel electrophoresis.

A cDNA synthesis referred to instructions of a TaKaRa Primer Script TMRT reagent kit with gDNA Eraser.

### (2) PCR Amplification

step 1: a PCR reaction solution (a 50-μl system) was prepared according to the following components in sequence: a 10× PCR Buffer (25 μl), ddH₂O (9 μl), dNTP (10 μl), GmSTK_IRAK-F (1.5 μl), GmSTK_IRAK-R (1.5 μl), CDNA (2 μl) and a KOD FX enzyme (1 μl);

Designed primers were as follows:

GmSTK_IRAK-F:
5'-CCTACCACATTAATTACTCACTCTTCACTCA-3'
GmSTK_IRAK-R:
5'-TCAACTTTAACGCTCATTCCTGCATTCAT-3';

step 2: a reaction was conducted on a BIO-RAD PTC-200 type PCR instrument and a reaction program was set as follows: denaturation at 94° C. for 2 min; then 98° C. for 10 sec, 55° C. 30 sec and 68° C. for 30 sec, a total of 33 cycles; then extension at 68° C. for 7 min; and storage at 4° C.; and

step 3: after a PCR product was recovered, connection to a PMD19-T vector (TaKaRa), transformation into Escherichia coli DH5α, blue-white spot screening, shaking, and sequencing were conducted and a sequence was shown in SEQ ID NO. 1.

### (3) Construction of Plant Over-Expressing Vector

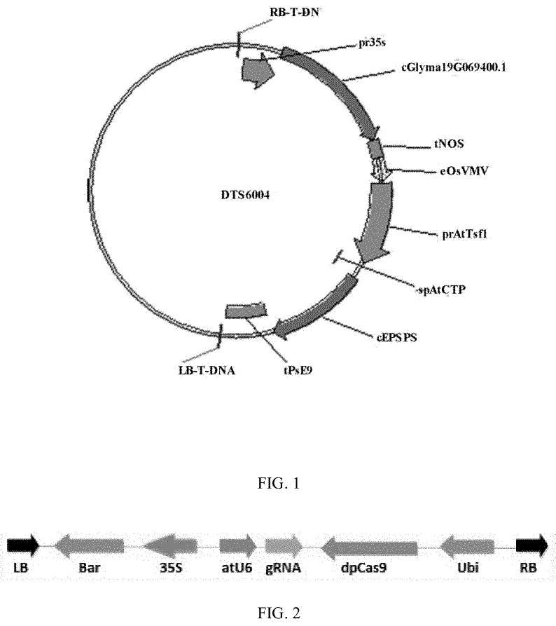

Homologous recombination linker primers were designed, a T vector containing the GmSTK_IRAK gene obtained in (2) was used as a template to amplify a full-length GmSTK_IRAK fragment with a recombination linker, the GmSTK_IRAK gene was forwardly introduced into a soybean expression vector pCAMBIA3300 by a seamless cloning technology, a recombinant plant expression vector DTS6004-GmSTK_IRAK was constructed, as shown in FIG. 1. The vector used an EPSPS gene as a transgene selection marker, the DTS6004-GmSTK_IRAK vector used a 35S promoter followed by a full-length GmSTK_IRAK cDNA, and GmSTK_IRAK over-expressing transgenic plants can be obtained after transgene.

The DTS6004 vector carries the selection marker gene EPSPS in a T-DNA region, the EPSPS encodes 5-enolpyruvyl shikimate-3-phosphate synthase (EPSPS), and the EPSPS can block an interference of glyphosate on a biosynthetic pathway and thus is not killed by the glyphosate.

Primers used for the seamless cloning were as follows:

An upstream primer:
5'-TTTGGAGAGAACACGTATGGCTGAGCTTCACTACCAAC-3'
A downstream primer:
5'-TCGGGGAAATTCGGGGTTAAGAAGCCTGCACCCCACTG-3'.

### (4) Construction of Plant Silenced Vector

GmSTK_IRAK gene information in a soybean reference genome in a soybean genome database (https://www.soybase.org) were referred to and an online website (http://crispr.hzau.edu.cn/CRISPR2/) was used to design objective targets of the gene. Design principles of the target were as follows: 1) knockout sites were located in a coding (CDS) region and as far as possible in the front end of a protein or an important functional domain; 2) transcripts with a higher proportion were covered as much as possible; 3) no off-targets existed or the off-targets were located in an intergenic region; 4) the targets with higher editing efficiency were optimized; and 5) a sequence had a relatively balanced GC content and was not easy to form a secondary structure. A target sequence was as follows:

gRNA: GCAGTACCTGTGAAGCACAA. According to an instruction in a CRISPR/Cas9 rapid construction kit VK005-04 (purchased from Beijing Viewsolid Biotech Co. Ltd), the gRNA was inserted into a gene editing vector to construct a CRISPR/Cas9 vector containing the GmSTK_IRAK target sequence, as shown in FIG. 2, where, the vector used a Bar gene as a transgene selection marker, used an AtU6 promoter followed by a Cas9 target sequence and a Cas9 gene, and was used for transgene to obtain GmSTK_IRAK-silenced transgenic plants.

### EXAMPLE 2

### Cultivation of GmSTK_IRAK Gene Over-Expressing And Silenced Transgenic Soybeans

### (1) Disinfection and Germination of Seeds

Surface disinfection of soybean seeds was conducted by using chlorine dry sterilization. Clean seeds that are mature and full, and free of disease spots and hard skins were selected and arranged in 90×15 mm culture dishes in a single layer manner. Lids of the culture dishes was opened, the culture dish was placed in a desiccator, a 500-ml glass beaker was placed in the desiccator, a 100-ml graduated cylinder was used to measure 75 ml of commercial bleaching water which was added into the beaker, a 10-ml graduated cylinder was used to measure 3 ml of 12 M HCl and the HCl was slowly added along a wall of the beaker. A lid of the desiccator was covered to ensure that the vessel was sealed, still-putting overnight was conducted for 10-16 h, after sterilization was completed, the culture dishes were covered and transferred to a sterile super clean bench, the lids of the culture dishes were opened, and strong wind blew for 25-40 min to remove residual chlorine. The disinfected seeds were sowed in a germination medium (GM) with umbilici facing down, the culture dishes were stacked and wrapped with preservative film, the culture dishes were placed in a biological incubator at 24° C. and incubation was conducted in the dark for 16-24 h.

### (2) Preparation of Agrobacterium

DNA of a recombinant vector DTS6004-GmSTK_IRAK plasmid was extracted, the recombinant vector was transformed into an Agrobacterium strain LBA4404 by an electrotransformation method and the Agrobacterium was stored in 50% glycerol. 2 days before transgene, 50 μl of the Agrobacterium glycerol containing the vector was pipetted into 5 ml of a YEP liquid medium supplemented with antibiotics ( 1/1000), and shake culture was conducted at 28° C. and 250 rpm for 24-36 h. 0.2-1 ml of saturated bacterial liquid was pipetted into a 250-ml YEP liquid medium supplemented with antibiotics ( 1/2000) for an enlarge cultivation to OD650 nm=0.8-1.0. The bacterial liquid was sub-packaged into several 50-ml sterile centrifuge tubes, centrifugation was conducted at 4,000 rpm and 25° C. for 10 min, colonies were collected and gently pipetted with 25-50 ml of a liquid co-cultivation medium (LCCM), and resuspension and precipitation were conducted for later use.

The YEP liquid medium: 10 g/L of peptone, 5 g/L of a yeast extract and 5 g/L of sodium chloride with a pH of 7.0;

The LCCM contains 1/10 of a B5 medium with major elements and trace elements and vitamins (Gamborg et al., 1968), 3% of sucrose and 3.9 g/L of an organic buffer agent 2(N-morpholine) ethanol sulfonic acid (MES) with a pH of 5.4, and sterilized at 120° C. for 20 min, and 0.25 mg/L of gibberellin (GA3), 1.67 mg/L of 6-benzyl adenine (BAP), 400 mg/L of cysteine (Cys), 154.2 mg/L of dithiothreitol (DTT) and 200 μmol/L of acetosyringone (As) were added in a sterile environment.

### (3) Preparation and Co-Cultivation of Explants

Swollen soybean seeds were placed on sterile absorbent paper, the seeds were cut longitudinally with a scalpel along the umbilici, cotyledons and a hypocotyl were evenly separated into two petals, and seed coats were removed for later use. An Agrobacterium resuspension was poured into clean sterile culture dishes, 50 explants were put, infection at room temperature were conducted for 20-30 min, the bacterial liquid was frequently stirred during the period, so that the explants fully contacted the fresh bacterial liquid. After the infection, the explants were taken out, blotted dry with sterile absorbent paper, and placed on a co-medium (CM) with sterile filter paper, and 7-10 explants were placed per dish with a paraxial side facing up and placed horizontally. The culture dishes were stacked and seal with preservative film, the sealed culture dishes were placed in a Percival incubator at 23° C., and a co-cultivation was conducted in the dark for 3-5 days.

A formula of the CM is the same as LCCM and 5 g/L of agar (Difco Agar, Noble) was added additionally.

### (4) Screening and Regeneration

After 3-5 days of the co-cultivation, the elongated hypocotyl was cut off to leave about 0.5 cm and the cut-off hypocotyl was inserted in a shoot induction (SI) medium with a screening agent at an oblique angle of 30-45°. A 3M breathable tape was used for sealing, the sealed medium was transferred to a culture room (24° C., 18/6 light intensity 140 μ moles/m²/sec), culture was conducted for 4 weeks, and a fresh SI medium was changed for use every two weeks. After 4 weeks of induction and screening of clustered shoots, the remaining cotyledons were removed, the clustered shoots were transferred to a shoot elongation (SE) medium, culture conditions were the same as an induction process of the clustered shoots, culture was conducted for 2-8 weeks, and a fresh SE medium was changed for use every 2 weeks. The shoots with an elongation of 3-4 cm were cut off, the cut-off shoots were dipped in indolebutyric acid (IBA) for 30 s-1 min, and the dipped shoots were inserted in a rooting medium (RM). After 1-2 weeks, when roots were about 2-3 cm long, rooting seedlings were taken out of the medium, the medium remaining on the roots were washed clean, and the rooting seedlings were transferred to the soil and moved to a greenhouse for cultivation. Cultivation conditions were 24° C. and 18/6 light intensity 140 μmoles/m²/sec.

The SI medium contains a B5 medium with major elements and trace elements and vitamins, 30 g/L of sucrose, 0.59 g/L of MES, and 8 g/L of agar (Sigma, USA). After sterilization at 120° C. for 20 min, 1.67 mg/L of BAP, 250 mg/L of ticarcillin (Tic) and 100 mg/L of cephalosporin (Cef) were added under sterile conditions.

The SE medium contains a MS medium with major elements and trace elements and vitamins (Murashige and Skoog, 1962), 30 g/L of sucrose, 0.59 g/L of MES and 8 g/L of agar (Sigma, USA) with a pH of 5.8. After sterilization at 120° C. for 20 min, 30.5 mg/L of GA, 50 mg/L of L-asparagine (L-Asp), 50 mg/L of glutamine (Glu), 0.1 mg/L of indoleacetic acid (IAA), 1 mg/L of zeatin (ZR), 250 mg/L of Tic and 100 mg/L of Cef were added under sterile conditions.

The RM medium contains a MS medium with major elements and trace elements and vitamins, 20 g/L of sucrose, 0.59 g/L of MES, 8 g/L of agar (Sigma, USA), 0.1 mg/L of IBA, 50 mg/L of L-Asp, 50 mg/L of Glu, 250 mg/L of Tic and 100 mg/L of Cef.

### EXAMPLE 3

### Validation of Transgenic Materials

Since a vector used for over-expressing transgenes contains a gene encoding 5-enolpyruvyl shikimate-3-phosphate synthase (EPSPS), the EPSPS can block an interference of glyphosate on a biosynthetic pathway and thus is not killed by the glyphosate. The herbicide glyphosate was used for identification, after a stock solution was diluted 1,000 times (with a concentration of 200 mg/L), the diluted glyphosate was used to spray transgenic seedling, negative plants withered and died, while positive plants showed obvious resistance and maintained good growth.

DNA from leaves of the surviving positive plants after the herbicide testing were extracted (by using a CTAB plant genomic DNA rapid extraction kit: Zoonbio Biotechnology Co., Ltd. and article number DN14-100T), and PCR was used to detect the EPSPS gene to further screen the positive materials. Sequences of primers of the EPSPS gene:

An upstream primer:
5'-AGGACGTCATCAATACGGGC-3'
A downstream primer:
5'-ATCCACGCCATTGAGCTTGA-3'

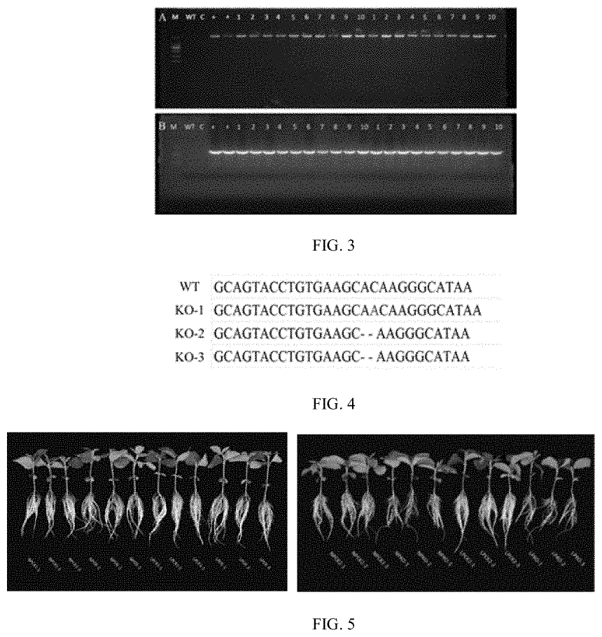

Finally, 10 independent transgenic lines of T0 generation were obtained. T1-generation seeds of the harvested transgenic materials were potted in sterilized mixed nutrient soil (nutrient soil: vermiculite=2:1) and placed in a greenhouse for cultivation. Cultivation conditions were 24° C. and 18/6 light intensity 140 μmoles/m²/ sec. At a V2 stage (when twoternately compound leave grew out), halves of the leaves were quick-frozen in liquid nitrogen, DNA was extracted, and fragments of the EPSPS gene were amplified by PCR to further screen the positive materials. According to FIG. 3B, the EPSPS gene can be detected in the transgenic materials.

A vector used for silencing transgenes contains a gene encoding phosphinothricin acetyl-CoA transferase (PAT), and the PAT can catalyze acetylation of free amino groups of phosphinothricin and thus inactivates the herbicide phosphinothricin. A herbicide Basta was used for identification, after a stock solution was diluted 1,000 times (with a concentration of 200 mg/L), the diluted Basta was used to spray transgenic seedling, negative plants withered and died, while positive plants showed obvious resistance and maintained good growth. DNA from leaves of the surviving positive plants after the herbicide testing were extracted (by using a CTAB plant genomic DNA rapid extraction kit: Zoonbio Biotechnology Co., Ltd. and article number DN14-100T), and PCR was used to detect a bar gene to further screen the positive materials. Sequences of primers of the bar gene:

An upstream primer:
5'-ATGAGCCCAGAACGACGC-3'
A downstream primer:
5'-ACGTCATGCCAGTTCCCGT-3'

The editing-type testing used DNA of the silenced plants as a template for PCR amplification, a PCR product was sequenced and used primer pairs were as follows:

An upstream primer:
5'-GCAGCAAATCCAAATCTACGAC-3'
A downstream primer:
5'-CGGTCTTCTCCTTTCGTCATATA-3'.

Sequence changes near a target sequence were shown in FIG. 4, where, KO-1 represented a nucleotide sequence of GmSTK_IRAK gene-silenced transgenic offsprings corresponding to an edited transgenic strain as shown in SEQ ID NO. 3 and KO-2 and KO-3 represented a nucleotide sequence of GmSTK_IRAK gene-silenced transgenic offsprings corresponding to an edited transgenic strain shown in SEQ ID NO. 4.

### EXAMPLE 4

Identification and determination of phenotypes related to low-phosphorus tolerance in soybeans (root architectures, biomass, phosphorus content, phosphorus absorption efficiency and yield)

### Identification of Root Architectures

Step 1: T3-generation transgenic seeds and wild-type seeds were planted in vermiculite by a hydroponic method; after five days of germination, the germinated seeds were transferred to ½ of a Hoagland nutrient solution. A phosphorus concentration was 0.005 μmol/L in a low-phosphorus treatment group and 5 μmol/L in a control group, a lack of potassium in the low-phosphorus treatment group was supplemented with the same amount of potassium chloride, and other nutrients remained unchanged.

Step 2: the nutrient solution was changed every three days in the hydroponic treatment, and after 10 days of the treatment, as shown in FIG. 5: over-expressing transgenic lines showed better growth under low-phosphorus stress conditions than wild types; and silenced transgenic lines showed significantly worsened growth under low-phosphorus stress conditions than the wild types.

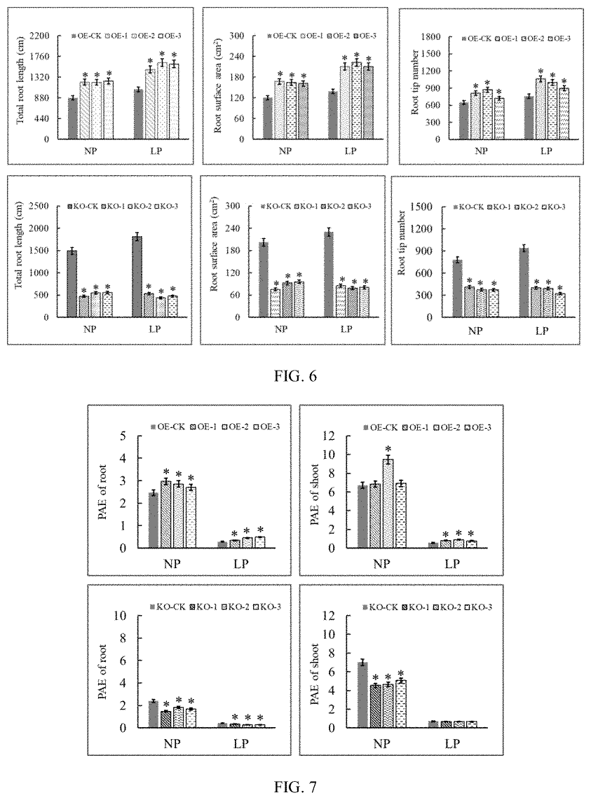

Step 3: after 10 days of the treatment, samples were taken to analyze root architectures with a root scanner. Results of the analysis were shown in FIG. 6: Compared with the wild types, total root length, root surface area and number of root tips of the over-expressing transgenic lines were significantly increased under normal- or low-phosphorus conditions; on the contrary, total root length, root surface area and number of root tips of the silenced transgenic lines were significantly reduced.

### Determination of Phosphorus Content and Phosphorus Absorption Efficiency

The samples (including Root and Shoot) after the root architecture analysis were put into a kraft paper bag and placed in an oven, enzyme-deactivated at 105° C. for 1 h, and dried at 60° C. to determine biomass for calculation of phosphorus absorption efficiency.

Step 1: about 0.1 g of the dried and ground plant samples (obtained through 0.25-0.5 mm sieves) were weighed, the weighed samples were put in digestion tubes (do not stick the samples on bottlenecks), a small amount of water dripped to moisten the samples, 5 mL of concentrated sulfuric acid was added, and the obtained mixture was shaken well (best placed overnight).

Step 2: the mixture was heated slowly on the dispelling furnace and a temperature was increased when the concentrated sulfuric acid decomposed and emitted a lot of white smoke.

Step 3: digestion was conducted until the solution was evenly brown-black, the digestion tubes were taken off to be slightly cooled, a bent-neck funnel was lifted, 10 drops of 30% of H₂O₂ dripped, and the digestion tubes continuously shaken.

Step 4: the solution was reheated (slightly boiled) for about 5 min, the reheated solution was taken off and slightly cooled, 5-10 drops of 30% of H₂O₂ dripped, and the digestion was conducted again.

Step 5: After the digestion was conducted until the solution was colorless or clear, the solution was reheated for 5-10 min (to drive off the remaining H₂O₂), the digestion tubes were taken off to be cooled, the funnel was rinsed with a small amount of water, and an eluate flows into the digestion tubes.

Step 6: the digestion solution was completely transferred to a 100-ml volumetric flask, a constant volume was set by using water and even shaking was conducted.

Step 7: 5 ml of the constant volume digestion solution was taken and a phosphorus concentration of the digestion solution was determined by using an AA3 continuous flow analyzer.

Step 8: the phosphorus absorption efficiency was calculated according to a formula: phosphorus absorption efficiency =phosphorus concentration/sample massxbiomass. Results were shown in FIG. 7: Compared with the wild types, the phosphorus absorption efficiency of roots of the over-expressing transgenic lines were significantly increased whether under normal- or low-phosphorus conditions and the phosphorus absorption efficiency of overground parts were significantly increased during a low-phosphorus treatment; the phosphorus absorption efficiency of roots of the silenced transgenic lines were significantly reduced and the phosphorus absorption efficiency of overground parts were also significantly reduced under normal-phosphorus conditions.

### Determination of Yield

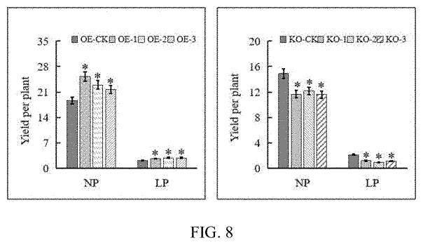

After maturity, single plants were harvested, the plants were dried and threshed, and grain weight was weighed with a one ten-thousandth balance as yield per plant. Results were shown in FIG. 8: Compared with wild-type controls, under normal-phosphorus conditions, yield per plant of the over-expressing transgenic lines were significantly increased, while the yield per plant of the silenced transgenic lines were significantly reduced; and compared with the wild-type controls, under low-phosphorus conditions, the yield per plant of the over-expressing transgenic lines were significantly increased, while the yield per plant of the silenced transgenic lines were significantly reduced.

The above descriptions are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. Use of a soybean protein kinase gene GmSTK_IRAK in improving phosphorus absorption and utilization efficiency of soybeans

2. The use according to claim 1, wherein, the soybean protein kinase gene GmSTK_IRAK has a nucleotide sequence shown in SEQ ID NO. 1 and a protein encoded by the soybean protein kinase gene GmSTK_IRAK has an amino acid sequence shown in SEQ ID NO. 2.

3. The use according to claim 1, wherein, the soybean protein kinase gene GmSTK_IRAK is over-expressed or silenced in soybean plants.

4. The use according to claim 3, wherein, the over-expression comprises introducing the soybean protein kinase gene GmSTK_IRAK into a target plant through a recombinant plasmid.

5. The use according to claim 4, wherein, the recombinant plasmid is a plant over-expressing vector containing the soybean protein kinase gene GmSTK_IRAK.

6. The use according to claim 5, wherein, the plant over-expressing vector is DTS6004.

7. The use according to claim 3, wherein, the gene GmSTK_IRAK in soybeans is silenced through a gene editing technology, and specifically, a Cas9 gene editing technology is used to edit the gene GmSTK_IRAK to obtain GmSTK_IRAK gene-silenced transgenic offsprings.

8. The use according to claim 7, wherein the GmSTK_IRAK gene-silenced transgenic offsprings have a nucleotide sequence shown in SEQ ID NO. 3 and/or SEQ ID NO. 4.

9. Use of a recombinant vector and cell containing a soybean protein kinase gene GmSTK_IRAK in improving phosphorus absorption and utilization efficiency of soybeans.

10. Use of a soybean protein kinase gene GmSTK_IRAK and a recombinant vector and cell thereof in breeding and cultivating soybeans.
